(19) United States
(12) Patent Application Publication
(10) Pub. No.: US 2025/0114502 A1
(43) Pub. Date: Apr. 10, 2025

(54) IMPLANTABLE MONITOR FOR DETERMINING FLUID STATUS IN DIALYSIS PATIENTS

(71) Applicant: William Frumkin, New York, NY (US)

(72) Inventor: William Frumkin, New York, NY (US)

(21) Appl. No.: 18/910,679

(22) Filed: Oct. 9, 2024

Related U.S. Application Data
(60) Provisional application No. 63/589,117, filed on Oct. 10, 2023.

(51) Int. Cl. A61M 1/16

(52) U.S. Cl. CPC A61M 1/1603; A61M 2205/3303

(57) ABSTRACT

A method of managing fluid status in patients having renal insufficiency or renal failure includes implanting a sensor device into a subject, to sense a physical parameter; comparing the physical parameter to a predetermined range to determine whether the physical parameter is within, above, or below the predetermined range and assign a score; applying a weighting factor to the score; and calculating a total score; and comparing the total score to at least one predetermined threshold to determine a fluid status of the subject. The sensor device has sensors that monitor the physical parameter. The method can be used in place of or in conjunction with the body weight and/or central venous pressure to predict fluid status, including fluid overload and dry weight.

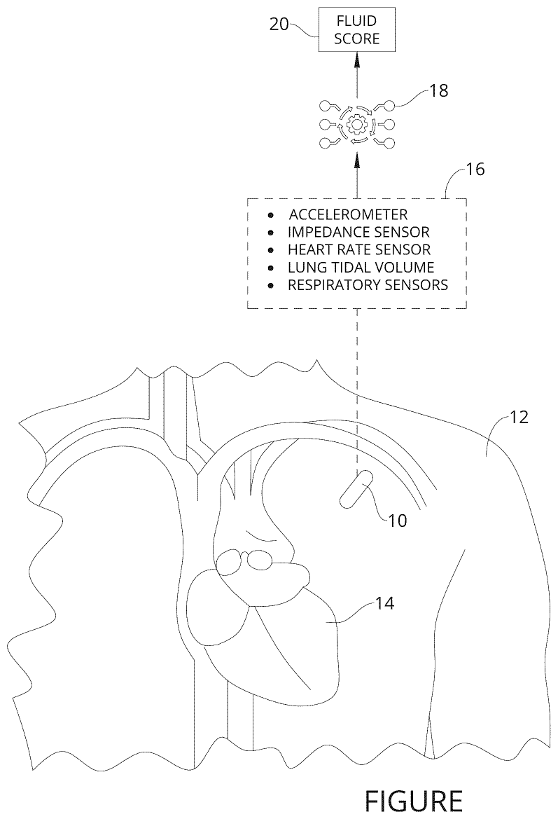

### CROSS-REFERENCE TO RELATED APPLICATION

This application claims the benefit of priority of U.S. provisional application No. 63/589,117, filed Oct. 10, 2023, the contents of which are herein incorporated by reference.

### BACKGROUND OF THE INVENTION

The present invention relates to determining fluid status in renal insufficient/renal failure patients and, more particularly, to an implantable sensor and predictive algorithm therefor.

Currently available methods of determining fluid status in renal failure patients, such as body weight and central venous pressure, are not constant measurements. These methods are often inaccurate in measuring fluid status as they do not account for changes in body weight or in venous pressure for other reasons. These inaccurate results lead to medical complications between treatments, such as passing out or experiencing an electrolyte imbalance. Moreover, the analysis is subjective.

As can be seen, there is a need for an accurate method of determining fluid status in patients with renal failure or renal insufficiency.

### SUMMARY OF THE INVENTION

In one aspect of the present invention, a method of managing fluid status in patients having renal insufficiency or renal failure comprises implanting a sensor device into a subject, wherein the sensor device has sensors operative to monitor a physical parameter; sensing the physical parameter using the implanted sensor; comparing the physical parameter to a predetermined range to determine whether the physical parameter is within, above, or below the predetermined range and assigned a score; applying a weighting factor to the score; and calculating a total score; and comparing the total score to at least one predetermined threshold to determine a fluid status of the subject.

The present invention provides a method of managing fluid status in renal failure patients. This method may be used in place of or in conjunction with the current practice of body weight and/or central venous pressure to predict fluid status, including fluid overload and dry weight.

These and other features, aspects and advantages of the present invention will become better understood with reference to the following drawings, description, and claims.

### BRIEF DESCRIPTION OF THE DRAWINGS

The sole FIGURE is a schematic view illustrating an implantable monitor according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a process of using an implantable battery powered device including sensors operative to obtain both chronic and acute measurements, both pre/post renal dialysis, to provide feedback and to develop a composite algorithm that accurately and objectively predicts fluid status in a renal insufficient or renal failure patient.

An implantable loop recorder is known for prediction of heart failure, such as the LUX-Dx II+™ ICM System from Boston Scientific. Applicant has discovered that an implantable loop recorder having selected sensors technologically improves the field of fluid status management in renal failure or renal insufficient patients.

The sensors may be selected from and are not limited to an accelerometer, a trans-thoracic impedance sensor, a heart rate sensor, lung tidal volume and respiratory sensors, and any combination thereof. These sensors can be included in a commercially available implantable cardiac monitor with a related clinic transmitter/remote home monitoring website to monitor and manage data. These measurements may be used to develop an algorithm to monitor fluid status more effectively to better guide dialysis treatments.

The implantable monitor and associated implantable kit may electronically communicate with a computer with a remote monitor to display real time fluid status data during dialysis treatments an enables remote monitoring for chronic changes. The fluid status data may be analyzed by a computer processor that runs an algorithm.

The implantable monitor may be used during dialysis to predict optimized dialysis treatments and to identify and/or predict complications. The monitor may be used to measure fluid status before, continuously during, and after dialysis. Additionally, the device may be used to monitor patient remotely, gathering data to predict and/or prevent complications via an algorithm. The algorithm may be used to create a score or risk factor, calculate a dialysis start and stop time, and determine an optimal time from one treatment to another to avoid adverse events.

Referring to the sole FIGURE, an implantable loop recorder 10 according to an embodiment of the present invention is shown implanted in a patient 12 proximal to the patient's heart 14. The loop recorder 10 comprises a plurality of sensors 16 selected from the group consisting of an accelerometer to detect changes in heart sounds to predict fluid overload, an impedance sensor (resistance across the chest) to look for fluid in the lungs, a heart rate sensor, a lung tidal volume sensor, respiratory sensors, and any combination thereof. A processor (not shown) receiving data from the sensors 16 analyzes the data by way of an algorithm 18 to calculate a fluid score 20.

It should be understood, of course, that the foregoing relates to exemplary embodiments of the invention and that modifications may be made without departing from the spirit and scope of the invention as set forth in the following claims.

What is claimed is:

1. A method of managing fluid status in patients having renal insufficiency or renal failure, comprising:
implanting a sensor device into a subject, wherein the sensor device has sensors operative to monitor a physical parameter;
sensing the physical parameter using the implanted sensor;
comparing the physical parameter to a predetermined range to determine whether the physical parameter is within, above, or below the predetermined range and assigned a score;
applying a weighting factor to the score; and
calculating a total score; and comparing the total score to at least one predetermined threshold to determine a fluid status of the subject.

2. The method of claim 1, further comprising:
iterating continuously the steps of sensing, comparing, applying, and calculating from initiation of dialysis; and
halting the dialysis when the total score falls within a second predetermined range.

3. The method of claim 1, further comprising:
iterating remotely the steps of sensing, comparing, applying, and calculating;
comparing the total score to a third predetermined range to predict if the subject will experience complications prior to a previously scheduled dialysis event; and
performing dialysis prior to a previously scheduled dialysis event if the prediction is positive for complications.

4. The method of claim 1, wherein the predetermined range, the weighting factor, and/or the at least one predetermined threshold are adjusted to improve an accuracy of the determination of the fluid status of the subject.

5. The method of claim 1, wherein the sensors are selected from the group consisting of: an accelerometer, a trans-thoracic impedance sensor, a heart rate sensor, a lung tidal volume sensor, a respiratory sensor, and any combination thereof.

6. The method of claim 1, wherein the physical parameter is selected from the group consisting of: a change in heart sounds, a presence of fluid in the subject's lungs, a heart rate, a lung tidal volume, breathing rate, airflow, cough frequency, body temperature, carbon dioxide level, and any combination thereof.
